(19) United States
(12) Patent Application Publication
(10) Pub. No.: US 2017/0119707 A1
(43) Pub. Date: May 4, 2017

(54) HOMEOPATHIC TOPICAL GEL FOR TRANSDERMAL DELIVERY OF COLCHICINE FORMULATIONS AND METHOD OF USE

(71) Applicant: GENSCO LABORATORIES, LLC, Miami, FL (US)

(72) Inventors: Carlos A. Alfaras, Miami, FL (US); Urbano Zamora, Davie, FL (US)

(21) Appl. No.: 15/408,164

(22) Filed: Jan. 17, 2017

Related U.S. Application Data
(63) Continuation-in-part of application No. PCT/US2015/040491, filed on Jul. 15, 2015.
(60) Provisional application No. 62/026,076, filed on Jul. 18, 2014.

(51) Int. Cl. A61K 31/165; A61K 9/00; A61K 47/18; A61K 47/24; A61K 47/20; A61K 47/14; A61K 41/00; A61K 9/06

(52) U.S. Cl. CPC A61K 31/165; A61K 41/0004; A61K 9/0014; A61K 9/06; A61K 47/24; A61K 47/20; A61K 47/14; A61K 47/18

(57) ABSTRACT

A homeopathic topical gel for transdermal delivery of colchicine formulations is disclosed. The colchicine formulations are effective in the treatment of constantly recurring acute gout flares. The homeopathic topical gels deliver an attenuated colchicine which has been produced using a degree of succussion and a level of water purity in excess of the guidelines for manufacturing homeopathic medicines set forth in the Homeopathic Pharmacopoeia of the United States.

### CROSS-REFERENCE TO RELATED APPLICATION

This application is a continuation-in-part of PCT/US2015/040491, filed on Jul. 15, 2015, which claims priority to U.S. Provisional Application Ser. No. 62/026,076, filed Jul. 18, 2014, the contents of all of which are hereby incorporated by reference in its entirety.

### FIELD OF THE INVENTION

The This application relates to a homeopathic topical gel for transdermal delivery of colchicine formulations. More particularly the application is directed toward colchicine formulations which are effective in the treatment of constantly recurring acute gout flares, and most particularly to homeopathic topical gels for transdermal delivery of an attenuated colchicine which has been produced using a degree of succussion and a level of water purity in excess of the guidelines for manufacturing homeopathic medicines set forth in the Homeopathic Pharmacopoeia of the United States (HPUS).

### BACKGROUND OF THE INVENTION

Pharmaceutical preparations in the form of topical gels useful for the administration of homeopathic agents are known in the art. For example, a transdermal gel marketed by Gensco Laboratories as SpeedGel Rx® is a prescription homeopathic topical analgesic gel that provides relief of pain and inflammation.

Colchicine has long been used to relieve acute gout attacks. It is a toxic natural product and secondary metabolite, originally extracted from plants of the genus Colchicum (autumn crocus, Colchicum autumnale, also known as “meadow saffron”). It was used originally to treat rheumatic complaints, especially gout, and still finds use for these purposes today despite dosing issues concerning its toxicity. It does not lower the level of uric acid, however in low doses, it does reduce the chance of future gout attacks by blocking the inflammation caused by uric acid crystals.

Side-effects include gastrointestinal upset and neutropenia. High doses can also damage bone marrow and lead to anemia and also cause hair loss. All of these side-effects can result from hyperinhibition of mitosis.

A main side-effect associated with all mitotic inhibitors is peripheral neuropathy, which is a numbness or tingling in the hands and feet due to peripheral nerve damage that can become so severe that reduction in dosage or complete cessation of the drug may be required. Microtubules are involved in vesicular transport. Peripheral nerves are among the longest in the body. Brownian motion is not significant enough in these peripheral nerves to allow vesicles to reach their destination. Thus, they are susceptible to microtubule toxins.

Although oral Colchicine may be an option to help reduce the number and severity of gout attacks that can result when uric acid levels change suddenly, long-term regimens of oral colchicine are absolutely contraindicated in patients with advanced renal failure (including those on dialysis), where cumulative toxicity is a high probability in this clinical setting. Additionally, Colchicine toxicity can be potentiated by the concomitant use of cholesterol-lowering drugs (statins, fibrates) resulting in severe neuromyopathy. This neuromuscular condition can be irreversible (even after drug discontinuation).

Oral colchicine has recently been approved by the FDA for treatment of acute gout flares, there is still a great deal of controversy surrounding its use as a long-term prophylactic treatment. The recommended dose of colchicine for acute gout is 1.2 mg at the first sign of symptoms followed by 0.6 mg one hour later. The maximum dose over a one hour period is 1.8 mg.

The recommended dose for preventing flares of gout in individuals older than 16 years of age is 0.6 mg once or twice daily. Most clinicians consider using colchicine for acute gout precisely in patients with contraindications to NSAIDs or prednisone, which are the usual first-line oral treatments. However the possibilities of adverse reactions either alone, or particularly as a result of potentiation by other commonly administered pharmaceuticals, remains a very real problem.

Therefore, there is a long felt need in the art for a low-dose topical colchicine formulation that is effective in the treatment of acute gout flares, while not exposing the patient to high systemic dosages of colchicine.

Deep U.S. Pat. No. 5,654,337, entitled “Topical Formulation For Local Delivery Of A Pharmaceutically Active Agent” relates to a composition useful in the delivery of pharmaceutically active agents through the skin. In one embodiment of the invention, the composition is formulated with a non-steroidal anti-inflammatory agent, such as ibuprofen or ketoprofen. Such formulation is rapidly absorbed through the skin to provide local relief from pain. In another embodiment of the invention, the composition is formulated with an antineoplastic agent. Such formulation is rapidly absorbed through the skin to provide local delivery to subcutaneous tumors. The composition is useful for transcutaneous delivery of other pharmaceutically-active compounds.

U.S. Published Patent Application 2014/0037718, entitled “Transdermal Pain Gel” discloses a transdermal gel including a complementary array of medicinal components which has beneficial effects for pain relief in muscular and connective tissues. The medicinal components include active ingredients having a synergistic effect for permitting musculoskeletal movement by countering the symptoms of musculoskeletal pain and being non-narcotic for avoiding dependency, and are combined in a liposomal base with a wetting agent to form a gel consistency suitable for skin application. The transdermal gel allows topical application of greater quantities and concentrations of the active ingredients than could safely be obtained via conventional oral administration.

Chinese Publication 103251550, entitled “Transdermal Delivery Ointment Containing Colchicine, And Preparation Method Thereof” provides a transdermal delivery ointment. The transdermal delivery ointment comprises 0.05-10 wt % of colchicine, 5-45 wt % of medical sterile water, 5-30 wt % of a water-soluble excipient, 10-60 wt % of an oil-soluble excipient, and 0.1-10wt % of a transdermal enhancer, wherein the sum of the percentages of the above components is 100 wt %. The invention also relates to a preparation method of the transdermal delivery ointment. The transdermal delivery ointment can be directly used against foci, and can avoid the generation of side effects of colchicine.

Chinese Publication 102366403A, entitled “Colchicine Microemulsion Transdermal Agent, Preparation Method Thereof And Application Thereof” discloses a colchicine microemulsion transdermal agent, a preparation method thereof and an application thereof. The colchicine microemulsion transdermal agent comprises lecithin, n-propanol, a surfactant, colchicine and an assistant. The colchicine microemulsion transdermal agent of the invention can be used for the preparation of drugs for treating gout. A microemulsion system of the colchicine microemulsion transdermal agent of the invention can promote colchicine to effectively permeate skins, so a purpose of gout treatment is reached, and toxic side effects of oral administration to livers and kidneys of human bodies are simultaneously reduced.

Maduri et al, in an article published in the Singapore Medical Journal, (Singapore Med J. 2012 November;53(11):750-4), entitled “Formulation of colchicine ointment for the treatment of acute gout” discloses substitute dosage forms of colchicine, specifically ointments to deliver the drug transdermally. Formulations having concentrations of 0.2% and 0.5% colchicine were evaluated for their effectiveness in delivering colchicine transdermally. Colchicine ointment was prepared using a self-formulated water-in-oil type of emulsion ointment base, with the colchicine dissolved in the water portion of the ointment base. Colchicine was found to be well-absorbed transdermally, although absorption was not 100%. No side effects were associated with its 0.2% formulation.

Hardevinder et al., in an article published in AAPS J. 2009 March; 11(1): 54-64 (published online 2009 Feb. 4), discloses an Elastic Liposomal Formulation for Sustained Delivery of Colchicine and evaluates its use in the treatment of gout.

None of the prior art teaches or suggests the treatment of acute gout with a homeopathically formulated colchicine containing transdermal gel.

### SUMMARY OF THE INVENTION

The presently disclosed invention is directed toward a homeopathic topical gel for transdermal delivery of colchicine formulations. More specifically, the application is directed toward colchicine formulations which are effective in the treatment of constantly recurring acute gout flares, and most particularly to homeopathic topical gels for transdermal delivery of an attenuated colchicine which has been produced using a degree of succussion and a level of water purity in excess of the guidelines for manufacturing homeopathic medicines set forth in the Homeopathic Pharmacopoeia of the United States.

Homeopathy is a system originated in the late eighteenth century, for the treatment of individuals afflicted with certain conditions or disease states, and involves the administration of minute doses of a substance, that in massive amounts may produce symptoms in healthy individuals similar to those of the disease itself. Homeopathy is based on the idea that substances that produce symptoms of sickness in healthy people will have a curative effect when given in very dilute quantities to sick people who exhibit those same symptoms. Homeopathic remedies are believed to stimulate the body's own healing processes.

One of the basic tenets of homeopathy is that small amounts of the substance are helpful, and that as the amount of the substance is increased, the less helpful and more deleterious the effect on the patient.

Homeopathic remedies are generally produced via iterated shaking (this shaking process is known as succussion) and dilution, in ethanol or in water, from a starting substance. The process of dilution and succussion leads to a gradual loss of chemical toxicity while gradually increasing the homeopathic potency; the more dilute remedies being of greater potency. This results in an attenuated product having enhanced efficacy at lower concentrations.

Accordingly, it is a primary objective of the instant invention to provide a homeopathic transdermal gel for delivery of an attenuated colchicine active ingredient to a patient experiencing an acute gout flare.

It is a further objective of the instant invention to teach a method for manufacturing a homeopathic transdermal gel containing an attenuated colchicine active ingredient to a patient experiencing an acute gout flare.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE DRAWINGS

FIG. 1 illustrates the results of 24 hour dosing over days 0-4;

FIG. 2A, 2B illustrate single dose kinetics results 1, 3, and 5 hours post dosing;

FIG. 3A, 3B and 3C illustrate results for repeated dosing 7 hours and 24 hours after the last dose;

FIG. 4 illustrates colchicine plasma concentrations at 1, 3 and 5 hours post dosing;

FIG. 5 illustrates the kinetics of colchicine plasma levels for Experiment 2;

FIG. 6 illustrates colchicine plasma concentrations at 1 hour and 24 hours post dosing;

FIG. 7 illustrates the kinetics of colchicine multi-dosing for Experiment.

### DETAILED DESCRIPTION

It is understood by the skilled artisan, that a definition of the term “about” normally acceptable in the pharmaceutical industry includes the range as stated, plus or minus amounts that are considered unlikely to have any detectable impact on formulation quality and performance. For example, the US Pharmacopeia allows a plus and minus range of 10% in the assay for the active ingredient in most solid dosage forms.

In an embodiment, the homeopathic transdermal colchicine gel formulation of the present invention contains about 12.6 weight percent of EPIKURON 130 P, about 8.9 weight percent of docusate sodium, about 17.8 weight percent of an attenuated colchicine supplied as Colchicinum 4X, about 12.6 percent isopropyl myristate, about 9.6 weight percent urea and about 38.5 weight percent purified water.

Colchicinum is defined as a Class B 1/100—Liquid class colchicine composition, containing 70% Alcohol (ethanol)—as designated by the HPUS. It is dissolved in 70% alcohol based on its solubility characteristics.

In accordance with established guideline for manufacture of Homeopathic medicines, an attenuation procedure is performed on the Colchicinum. The attenuation process includes serial dilutions with defined periods of succession, which involve vigorous mechanical agitation and blending of the liquid for a defined period of time, between each dilution. The minimum successive requirement of the HPUS is 10 repetitions.

The attenuation process carried out follows the methodology of homeopathic dilution. This is a process in which a substance is diluted with either alcohol or distilled water and then vigorously mixed. Homeopaths developed a decimal scale (D or X), diluting the substance to ten times its original volume for each stage. Therefore, a 1X solution is a 1:10 dilution of the base solution; 2X is a 1:100 dilution; 3X is a 1;1000 dilution; and a 4X (the base concentration for Colchicinum used in COLCIGEL™) is a 1:10,000 dilution. So, if the original base solution was at a concentration of 1 gm/1 ml, a 1:10,000 dilution would yield a 0.0001 gm/1 ml or in different units, 100 mcg/1 ml solution.

In the case of COLCIGEL™ the total amount of Colchicinum 4X used is 17.8% of the base, therefore COLCIGEL™ contains 17.8 mcg/1 gram of total product or 0.00178% of the total weight of the product.

The specific procedure for forming the attenuated Colchicinum of the present invention is as follows:

One part (by weight) of Colchicinum is dissolved in a sufficient quantity of 70% alcohol (the alcohol is ethyl alcohol) to produce 10 parts by volume and repeatedly succussed, in excess of the HPUS minimum requirements, producing a formation equivalent to Colchicinum 2X.

Subsequently, one volume of the Colchicinum 2X is added to 9 volumes of 70% alcohol and repeatedly succussed, in excess of the HPUS minimum requirements, yielding Colchicinum 3X.

Lastly, one volume of the Colchicinum 3X is added to 9 volumes of 70% alcohol and repeatedly succussed, in excess of the HPUS minimum requirements, yielding the attenuated Colchicinum 4X used in the topical gel.

Definitions

In accordance with the present invention, the term “transdermal colchicine gel formulation” is directed toward a formulation which does not contain colchicine, per se, but rather, it is the homeopathic version, colchicinum, which is a natural, diluted form of colchicine.

In accordance with the present invention, the term de-oiled, powdered soybean lecithin enriched with about 30% phosphatidylcholine refers to a product such as EPIKURON 130 P, available from Cargill Corporation.

In accordance with the present invention, Dioctyl sodium sulfosuccinate or docusate sodium is an ionic surfactant, and is used as an emulsifying, wetting, and dispersing agent.

In accordance with the present invention, Isopropyl myristate (Propan-2-yl tetradecanoate) is the ester of isopropanol and myristic acid. Isopropyl myristate is used in cosmetic and topical medicinal preparations where good absorption through the skin is desired.

In accordance with the present invention, Urea or carbamide is an organic compound with the chemical formula CO(NH2)2. The molecule has two —NH2 groups joined by a carbonyl (C═O) functional group.

In accordance with the present invention, the purified water used in the preparation of the formulation has a level of total organic carbon (TOC) of about 300 ppb, which is substantially higher in purity than the 500 ppb required by the HPUS.

In an embodiment, the transdermal colchicine gel formulation is formed in accordance with the following steps:
1. Add about 12.60 kg isopropyl myristate to a 40 gallon stainless steel tank, and begin operation of a Lightnin Air Mixer;
2. Slowly add about 12.60 kg of Epikuron 130 P to the 40 gallon stainless steel tank from Step #1, and continue to mix until dissolved;
3. Add about 8.90 kg docusate sodium to the container of step #1 and continue to mix until dissolved;
4. Separately, place purified water, in an amount of about 38.5 kg into a 55 gallon plastic drum and begin mixing with a Gast air mixer;
5. Add about 9.60 kg urea to the 55 gallon plastic drum of step #4 and continue to mix until dissolved;
6. Add about 17.8 kg Colchicinum 4X into the 55 gallon plastic drum and mix until dissolved;
7. Charge all of the ingredients in the 40 gallon stainless steel tank to the 55 gallon plastic drum, and mix for 30 minutes.

The resulting topical gel contains approximately 20 μg/ml, and is identified herein as Gensco Colchicine topical gel (COLCIGEL™)

Experimental Protocol

The objective of this experiment was to measure the serum level of a homeopathic transdermal colchicine gel formulation as described herein when applied as a gel to the skin using rabbit models for study. The approved drug colchicine is used to treat disease states such as gout and dermatitis. The transdermal application of colchicine provides a localized treatment for such disease states. There is no currently approved or marketed topical formulation for gout available in the US. It would be desirable to demonstrate that very low doses of colchicine administered transdermally at the site of a gout flare could provide symptomatic relief without significant systemic absorption and, therefore, the associated drug interactions, adverse effects, and toxicities seen with traditional oral dosing of colchicine. This experiment will result in a controlled measurement of the level of colchicine that enters the circulation via this novel transdermal delivery system. In rabbits, we have performed two types of studies to ascertain 1) the steady-state levels of drug in the serum with repeated application, and 2) the single-dose kinetics of serum drug levels over time. Simultaneously, we have assessed the skin for irritation.

Methodology

New Zealand White (Oryctolagus cuniculus) rabbits were chosen. The relative diffusion of drug agents across the rabbit skin and the sensitivity of the rabbit skin to topical agents has been calibrated relative to the human after extensive use of this model for transdermal drug delivery. Further, blood collection is relatively simple in this model and the species requires no specialized care. The rabbits in the 2.5-3 kg range have 150-180 ml of whole blood. All experiments and handling of the rabbits were performed with oversight and approval by the University of Alabama

Institutional Animal Care and Use Committee.

Dosing: Gensco Colchicine topical gel (COLCIGEL™): 20 μg/ml (0.002%). One pump=approximately 125 μl (2.5 μg). Sodium Lauryl Sulfate: 0.1% in water. Sodium Lauryl Sulfate is a known positive control, is used as a control agent in human clinical trials, and will not cause more than slight skin irritation or discomfort (CDER Guidance for Industry # 2887FNL)

Experiment 1 (24 h Repeat Dosing; Single Pump Colchicine Gel and Skin Irritation Assessment):

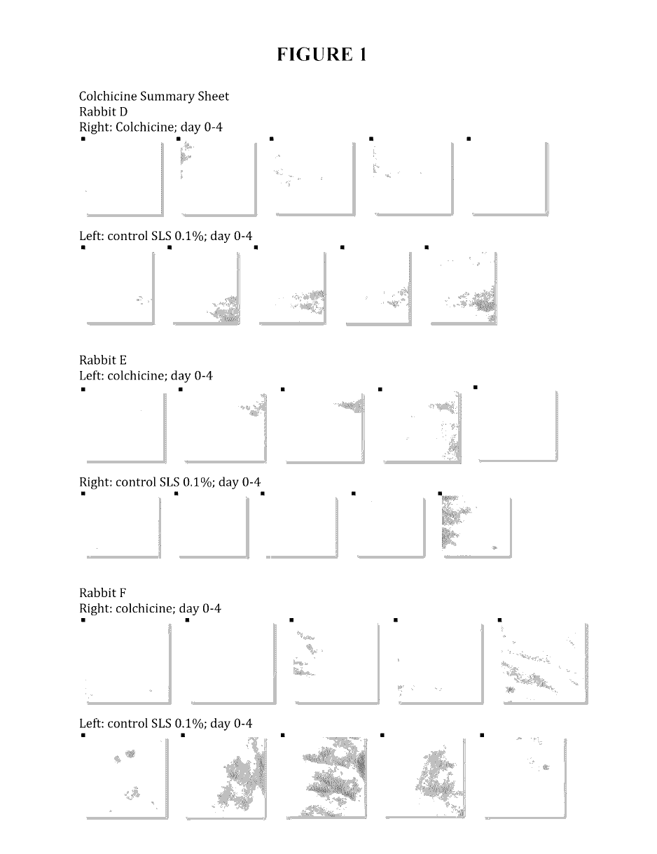

After animal adaptation, the first experiment was a repeated daily dose experiment for 4 days. The total “n” for this experiment is 3. All rabbits were sedated for shaving and application of a chemical depilatory cream (Veet) to remove the hair from the mid-dorsum. After application of the cream for 60-120 seconds, the cream and hair were removed with moist gauze. All animals received 1 pump (125 μl) of colchicine gel (COLCIGEL™) on one side of the midline, and 200 μl of a control irritant, 0.1% sodium lauryl sulfate, on the other side, each within a 2.5 cm×2.5 cm region. The treatments were applied on day 0, 1, 2, 3. Arterial blood was collected daily prior to treatment on day 1, 2, 3, 4. FIG. 1 illustrates photographic images of the application sites on days 0-4.

Experiment 2 (Single-Dose Kinetics):

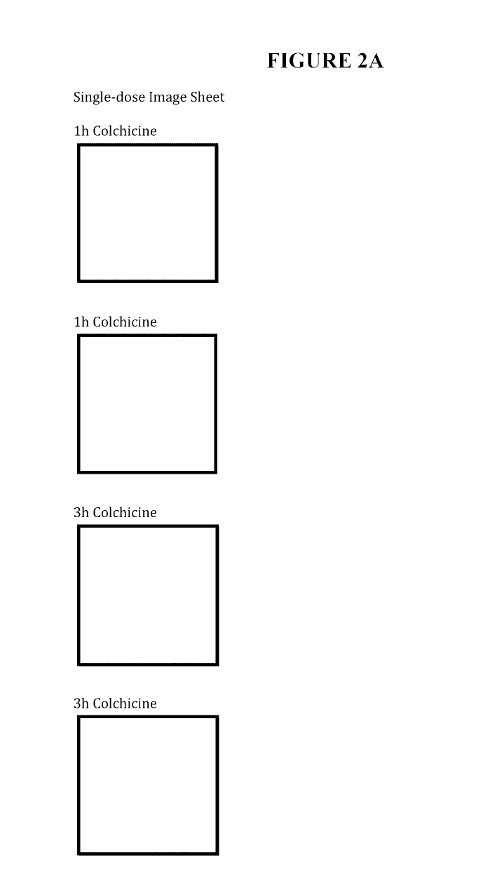

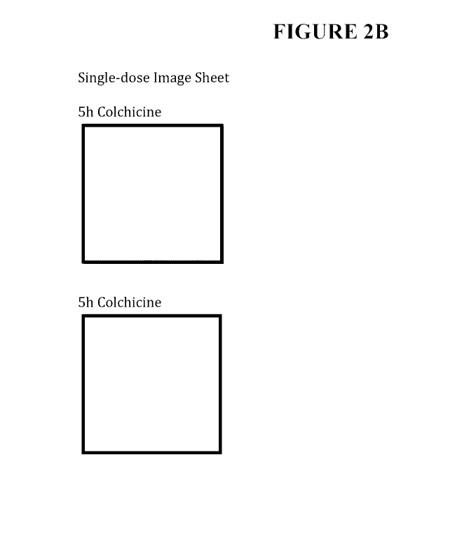

Due to the low levels of colchicine measured in the blood of the rabbits each day in Experiment 1, the single-dose kinetics were measured 1 h, 3 h, and 5 h after a single 375 μl A dose of colchicine gel were applied to a 2.5 cm×2.5 cm patch on opposite sides of the shaved rabbit dorsum. Six anesthetized rabbits had the hair on the dorsum clipped and a pre-treatment arterial blood draw. Six rabbits then received the test gel at time 0 and two rabbits had blood drawn after 1 h, two others had blood drawn after 3 h, and the last 2 rabbits had blood drawn after 5 h. FIGS. 2A-2B are photographic images made of each site at each time point when arterial blood was collected.

Experiment 3:

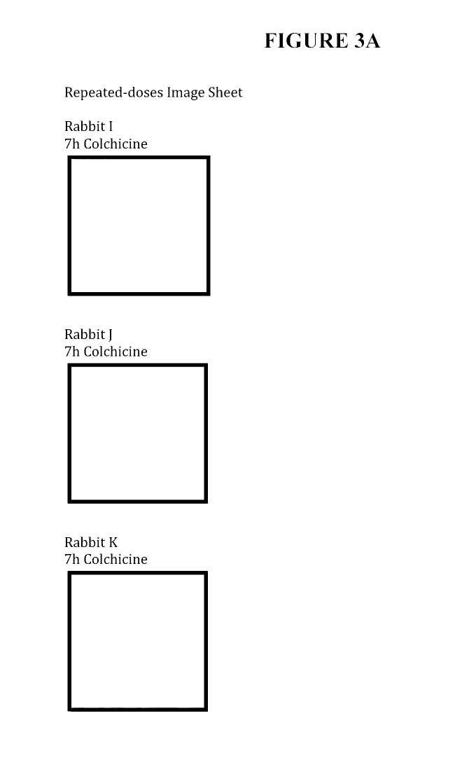

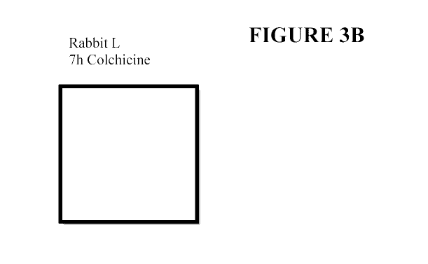

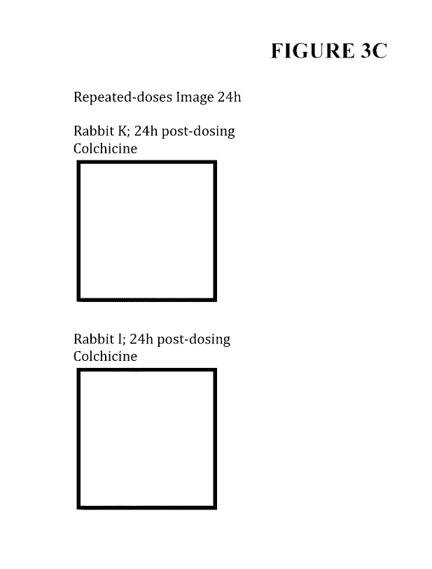

Four anesthetized rabbits had the hair on the dorsum clipped and a pre-treatment arterial blood drawn. All rabbits were treated at time 0 with a 375 μl dose of colchicine gel applied to a 2.5 cm×2.5 cm patch on opposite sides of the shaved rabbit dorsum. The application of the gel was repeated after 3 hours and again after 3 additional hours. One hour following the last gel application, arterial blood was collected from the 4 rabbits sequentially during the next 50 minutes and the treatment sites were photographed. After 24 h, arterial blood was again collected and the sites were photographed. The results are illustrated in FIGS. 3A-3C.

Assay:

All arterial blood samples were approximately 1 ml and were collected in syringes coated with sodium heparin and stored in tubes containing 20 μl sodium heparin. All blood samples were centrifuged at 2,000×g for 17 minutes and the plasma was removed to a fresh tube and immediately frozen at −80 ° C. Plasma samples were kept frozen until preparation for assay. A protocol was devised using tandem HPLC/MS separation and quantification to measure colchicine in a sample.

Samples were analyzed by a mass spectrometry/HPLC combination method that determined plasma concentration relative to standard curves. Samples were assayed in each batch with freshly prepared calibration samples of 5 concentrations in duplicate and quality control samples in duplicate at the high, mid, and low expected ranges. Acceptable analytical runs had standard curves where at least 6 of 10 individual standards from each curve met method criteria. Deviations of the back-calculated standards were required to be within T1/2a 25% of the nominal concentration. The calculated concentrations of acceptable analytical quality control samples were also required to be within ±25% of the nominal concentration.

Skin Irritation:

Photographs of the test and control sites were made under standardized lighting, focal length, and magnification. Photographic images were made just prior to application of a subsequent dose. Pre-treatment images were compared to post-treatment images of experimental sites and, in Experiment 1, control sites where the irritant 0.1% SLS was placed. The dermal response was scored as follows for each image.

I. Dermal Response:
0=no evidence of irritation
1=minimal erythema, barely perceptible
2=definite erythema, readily visible; minimal edema or minimal popular response
3=erythema and papules
4=definite edema
5=erythema, edema, and papules
6=vesicular eruption
7=strong reaction spreading beyond test site

II. Other Effects:
A=slight glazed appearance
B=marked glazing
C=glazing with peeling and cracking
F=glazing with fissures
G=film of dried serous exudate covering all or part of the delivery site
H=small petechial erosions and/or scabs

Results

Experiment 1 (24 h Dosing of Colchicine Gel)

No detectable colchicine was measured after daily single dose application in any of the 3 experimental rabbits (data not shown). These results suggested that either none of the 2.5 μg is per pump was absorbed into the circulation, or that more of the drug was absorbed but metabolized within the 24 h period.

There was minimal skin irritation caused by the colchicine gel from day 1-4 of application (mean dermal score 0.4±0.5) as illustrated in FIG. 1.

Experiment 2 (Single-Dose Kinetics Colchicine Gel)

The colchicine was rapidly absorbed (T1/2a=30 min), almost all (90%) of the 7.5 μg dose entered the bloodstream reaching a maximal concentration of 0.26 ng/ml±0.09, and it was rapidly eliminated (T1/2e=65 min) with none detectable 5 h after application.

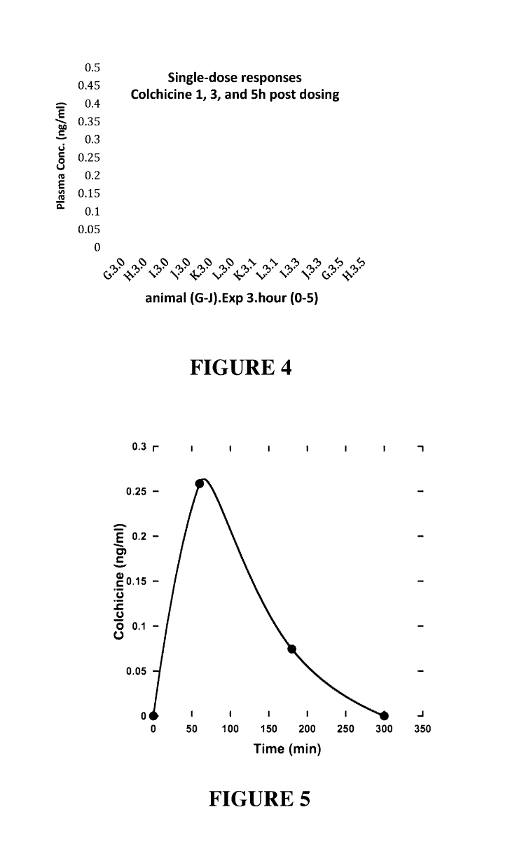

No skin irritation was detected over the 5 h application period, as illustrated in FIGS. 2A-2B. Colchicine plasma concentrations at 1, 3 and 5 hours post dosing are illustrated in FIG. 4.

The kinetics of colchicine plasma levels are illustrated in FIG. 5:
T1/2a=30 min
Absorption rate constant (ka)=0.693/0.5 h=1.4/h
T1/2e=65 min
Elimination rate constant (ke)=0.693/1.1 h=0.64/h
Total plasma absorbance=90% (AUC)
Cmax=0.26 ng/ml±0.09 ng/ml
Tmax=60 min

In an adult human, this same amount of gel (375 μl) would result in a maximal concentration of 9 pg/ml colchicine in the blood due to the larger blood volume.

Experiment 3: Drug Accumulation by Multi-dosing

Following three dosing events administered every 3 hours using 375 μl of gel per dose, colchicine plasma levels were measured at peak levels 1 hour following the third dose and were less than 0.45 ng/ml in all 4 rabbits. As it took an additional 15, 35, and 55 minutes to collect blood from rabbits J-L, the measured plasma levels of colchicine dropped progressively from 0.42 ng/ml to 0.2 ng/ml within the hour, as expected from the single-dose elimination kinetics.

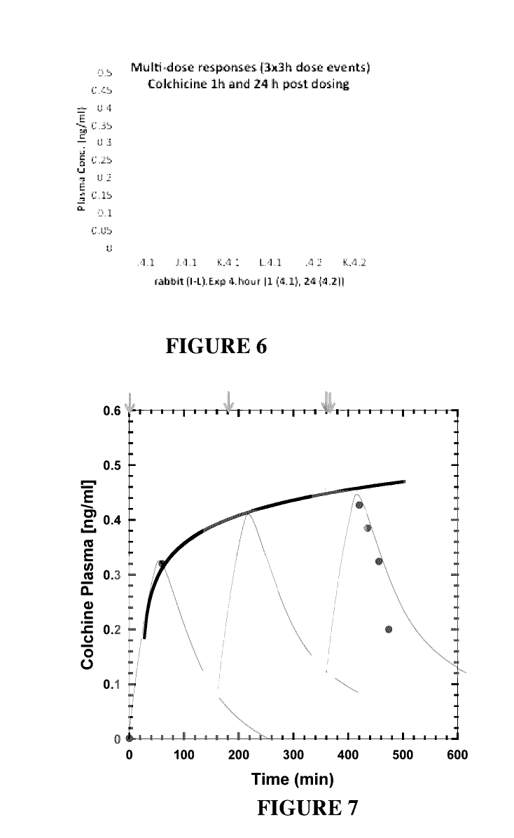

No skin irritation was measured after 3 doses over 7 h of colchicine gel application to the skin (rabbits I-L). No erythema was detected 24 h after dosing (rabbits I and K). Results are illustrated in FIGS. 3A, 3B and 3C. Colchicine plasma concentrations at 1 hour and 24 hours post dosing are illustrated in FIG. 6.

Kinetics of colchicine multi-dosing are illustrated in FIG. 7 wherein Arrows indicate dosing events. Double arrow indicates final dose. Single-dosing kinetic curves overlay the data, and the bold line is logarithmic progression prediction built to data:

With elimination exponentially progressing over a 5 h period, 3 h repeated dosing led to some accumulation of colchicine in the blood with repeated dosing. From the logarithmic progression analysis of these data it appears that 3 h dosing would approximate a steady state level of 0.5 ng/ml after 5 sequential administrations. After the third and final dose administration at 360 min (double arrow) the plasma levels fell quickly during the animal handling time, as expected from the single dose data. Given the kinetics of the single dose, it appears that 3 h repeated dosing of the colchicine gel provides a proper and safe maintenance dose regime.

Summary Statement

These data demonstrate that the colchicine from the Gensco COLCIGEL™ topical colchicine gel formulation is absorbed relatively rapidly, within 60 minutes, almost completely (90%), and safely. The elimination of these drugs once absorbed is relatively rapid, within 6 hours.

These data suggest that the Gensco COLCIGEL™ topical colchicine gel may be applied safely in a repeated fashion every 3 hours where blood levels of the drug would remain at safe levels and the skin would be expected to experience no irritation or, at most, mild erythema, in association with the lidocaine gel.

Study Highlights

In this study of the colchicine topical gel (COLCIGEL™) the gel was found to be safe and suitable for repeated application to the skin. The COLCIGEL™ was 90% absorbed into the bloodstream, reaching peak blood levels of 260 picograms per milliliter in a rapid 60 minutes after a single 375 microliter dose (3 pumps). The colchicine was rapidly eliminated from the blood within 5 hours, but dosing every 3 hours was able to sustain the blood levels allowing a predicted 500 picograms per milliliter to be maintained. There was little to no skin irritation resulting from prolonged application of the colchicine gel over a period of 4 days.

The safety demonstrated in these rabbits translates to safety in humans for the COLCIGEL™. The measured blood levels would be lower (approximately 28 fold) in the human. Further, because the rabbit skin is slightly more delicate than the human, the acceptability for repeated application of the COLCIGEL™ in humans was fully validated.

In an embodiment, the present invention contemplates a method for the treatment of an acute gout flare, in a human, whereby serial administration of an effective amount of a homeopathic transdermal colchicine gel formulation, e.g. about a 375 microliter dosage form, is administered at the site of the gout flare, utilizing a 3 hour repetitive dosage regimen, whereby this dosage regimen is effective to maintain blood levels of about 15-20 picograms/ml of colchicinum.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A homeopathic transdermal colchicine gel formulation, useful in the treatment of acute gout flares, comprising:
about 12.6 weight percent of a de-oiled, powdered soybean lecithin enriched with about 30% phosphatidylcholine;
about 8.9 weight percent of docusate sodium;
about 17.8 weight percent of attenuated colchicine, wherein said attenuated colchicine is Colchicinum 4X, whereby the total amount of colchicinum in the formulation is about 0.00178%;
about 12.6 percent isopropyl myristate;
about 9.6 weight percent urea; and
about 38.5 weight percent purified water.

2. The homeopathic transdermal colchicine gel formulation of claim 1, wherein said purified water contains a level of total organic carbon (TOC) of about 300 ppb.

3. A process for forming a homeopathic transdermal colchicine gel formulation comprising:
1. adding about 12.60 kg isopropyl myristate to a first mixing vessel;
2. adding about 12.60 kg of a de-oiled, powdered soybean lecithin enriched with about 30% phosphatidylcholine to said first mixing vessel, and continuing to mix until dissolved;
3. adding about 8.90 kg docusate sodium to said first mixing vessel and continuing to mix until dissolved;
4. separately, placing purified water, in an amount of about 38.5 kg into a second mixing vessel and initiating mixing with an air mixer;
5. adding about 9.60 kg urea to said second mixing vessel and continuing to mix until dissolved;
6. adding about 17.8 kg attenuated colchicine, wherein said attenuated colchicine is Colchicinum 4X, to said second mixing vessel and continuing to mix until dissolved; and
7. charging all of the ingredients from said first mixing vessel to said second mixing vessel, and mixing for 30 minutes;
whereby the total amount of colchicinum in said homeopathic transdermal colchicine gel formulation is about 0.00178%.

4. The process of claim 3, wherein said purified water contains a level of total organic carbon (TOC) of about 300 ppb.

5. A method for the treatment of an acute gout flare comprising:
administration of a homeopathic transdermal colchicine gel formulation at a site of a gout flare, wherein said homeopathic transdermal colchicine gel formulation comprises about 12.6 weight percent of a de-oiled, powdered soybean lecithin enriched with about 30% phosphatidylcholine, about 8.9 weight percent of docusate sodium, about 17.8 weight percent of attenuated colchicine, wherein said attenuated colchicine is Colchicinum 4X, thereby providing a total amount of colchicinum in said formulation of about 0.00178%, about 12.6 percent isopropyl myristate, about 9.6 weight percent urea and about 38.5 weight percent purified water;
whereby symptomatic relief of said acute gout flare is provided without significant systemic absorption.

6. The method for the treatment of an acute gout flare in accordance with claim 5, wherein said purified water contains a level of total organic carbon (TOC) of about 300 ppb.

7. The method in accordance with claim 5, wherein said homeopathic transdermal colchicine gel formulation is administered by serial administration of an effective amount of a homeopathic transdermal colchicine gel formulation, utilizing a 3 hour repetitive dosage regimen, whereby said dosage regimen is effective to maintain blood levels of about 15-20 picograms/ml of colchicinum.

8. The method of claim 7, wherein said effective amount of said homeopathic transdermal colchicine gel formulation is about a 375 microliter dosage form.
